(19) United States
(12) Patent Application Publication
(10) Pub. No.: US 2009/0023925 A1
(43) Pub. Date: Jan. 22, 2009

(54) N'-(2-HALOBENZYLIDENE)SULFONYLHYDRAZIDES AS INTERMEDIATES IN THE MANUFACTURE OF ARYLSULFONYLINDAZOLES

(75) Inventors: Anita Wai-Yin Chan, Wayne, PA (US); Mahmut Levent, Bloomfield, NJ (US); David Place, Warwick, NY (US); Panolil Raveendranath, (US); Sanjay Raveendranth, Monroe, NY (US); Vijay Raveendranath, Albany, NY (US); Girija Raveendranath, Monroe, NY (US); Xinxu Shi, Flushing, NY (US); Charles Wu, Denville, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/147,586

(22) Filed: Jun. 27, 2008

Related U.S. Application Data
(60) Provisional application No. 60/937,648, filed on Jun. 28, 2007.

(51) Int. Cl. C07D 241/04; C07D 405/10

(52) U.S. Cl. 544/371; 544/399

(57) ABSTRACT

The present invention provides N′-(2-halobenzylidene)sulfonylhydrazides of formula I

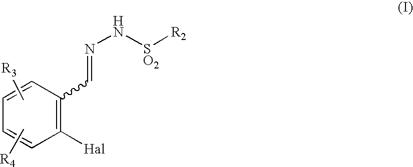

Compounds of formula I are useful for the manufacture of 1-arylsulfonylindazole 5-HT6 ligands.

### CROSS-REFERENCE TO RELATED APPLICATIONS

This application claims the benefit of U.S. Provisional Application No. 60/937,648, filed Jun. 28, 2007, the contents of which are incorporated herein by reference in their entirety.

### FIELD

The present invention relates to N′-(2-halobenzylidene)sulfonylhydrazide compounds and their use in the manufacture of 5-HT6 ligands.

### BACKGROUND

Arylsulfonylindazoles are an important class of 5-hydroxytryptamine-6 (5-HT6) ligands useful in the treatment of central nervous system (CNS) disorders related to or affected by the 5-HT6 receptor, such as cognitive disorders or anxiety disorders. Novel 1-arylsulfonylindazole compounds and their use as 5-HT6 ligands are described in U.S. Pat. No. 7,034,029; U.S. Pat. No. 6,815,456; U.S. Pat. No. 6,831,094; and U.S. Pat. No. 6,509,357. Said 1-arylsulfonylindazole compounds are generally prepared via the reduction of an ortho-nitrotoluene derivative by catalytic hydrogenation to give the corresponding amine; reacting said amine with a nitrite reagent, such as isoamylnitrite, to give an indazole intermediate; and reacting said indazole with an arylsulfonyl halide. However, on a manufacturing scale, a process route which utilizes nitro compounds, nitrite reagents and a hydrogenation step is undesirable due to safety, environmental and economic concerns.

Therefore, it is an object of this invention to provide an intermediate compound useful in the process of manufacture of a 1-arylsulfonylindazole 5-HT6 ligand.

It is another object of this invention to provide an effective and efficient process for the manufacture of a 1-arylsulfonylindazole 5-HT6 ligand which avoids the use of a nitro compound, a nitrite reagent and a hydrogenation step.

Other objects and features of the invention will become more apparent from the detailed description set forth hereinbelow.

### SUMMARY

The present invention provides a compound of formula I

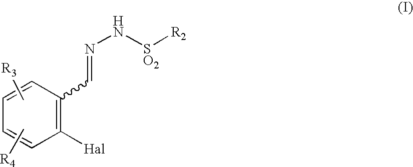

wherein
Hal is Cl, Br or I;
R₂ is an optionally substituted aryl or optionally substituted heteroaryl group;
R₃ and R₄ are each independently H, NR₅R₆, OR₇, or an optionally substituted alkyl group;
R₅ and R₆ are each independently H or an optionally substituted alkyl group, or R₅ and R₆ may be taken together with the atom to which they are attached to form an optionally substituted 5- to 7-membered ring optionally containing an additional heteroatom selected from NR₈, O or S;
R₇ is H or an optionally substituted alkyl group;
R₈ is H, COR₉, CO₂R₉, Si(R₁₀)₃ or an optionally substituted alkyl group; and
R₉ and R₁₀ are each independently an optionally substituted alkyl group;
or the E and Z isomers thereof.

Also provided is the use of a compound of formula I in the manufacture of a 1-arylsulfonylindazole 5-HT6 ligand. In particular, the invention provides a process for the manufacture of a compound of formula IV

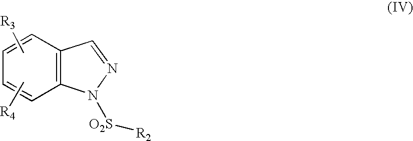

wherein
R₂, R₃ and R₄ are as defined above;
   which process comprises reacting a compound of formula I

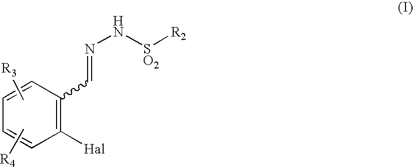

wherein R₂, R₃, R₄ and Hal are as described hereinabove, with a base in the presence of a copper containing catalyst in the presence of a solvent.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

The ability of the 5-hydroxytryptamine-6 (5-HT6) receptor to bind a wide range of therapeutic compounds used in psychiatry, coupled with its intriguing distribution in the brain has stimulated significant interest in compounds which are capable of interacting with or affecting said receptor. Compounds known to be 5-HT6 ligands include 1-arylsulfonylindazole compounds such as those described in in U.S. Pat. No. 7,034,029; U.S. Pat. No. 6,815,456; U.S. Pat. No. 6,831,094; and U.S. Pat. No. 6,509,357. Heretofore, said 1-arylsulfonylindazole compounds were prepared via the reduction of an ortho-nitrotoluene derivative by catalytic hydrogenation to give the corresponding amine; reacting said amine with a nitrite reagent, such as isoamylnitrite, to give an indazole intermediate; and reacting said indazole with an arylsulfonyl halide. However, on a manufacturing scale, a process route which utilizes nitro compounds, nitrite reagents and a hydrogenation step is undesirable due to safety, environmental and economic concerns. An alternative process route, which avoids the use of nitro compounds, nitrite reagents and a hydrogenation step, is highly desired.

Surprisingly, it has now been found that an N′-(2-halobenzylidene)-sulfonylhydrazide compound of formula I may be used in the manufacture of a 1-arylsulfonylindazole 5-HT6 ligand. Advantageously, the use of said N′-(2-halobenzylidene)sulfonylhydrazide in the manufacture of 1-arylsulfonylindazole 5-HT6 ligands avoids the use of nitro compounds, nitrite reagents and a hydrogenation step. Accordingly the present invention provides a compound of formula I

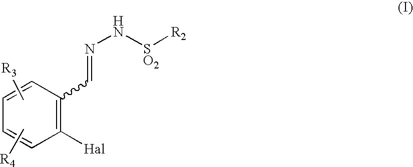

wherein
Hal is Cl, Br or I;
R₂ is an optionally substituted aryl or optionally substituted heteroaryl group;
R₃ and R₄ are each independently H, NR₅R₆, OR₇, or an optionally substituted alkyl group;
R₅ and R₆ are each independently H or an optionally substituted alkyl group or R₅ and R₆ may be taken together with the atom to which they are attached to form an optionally substituted 5- to 7-membered ring optionally containing an additional heteroatom selected from NR₈, O or S;
R₇ is H or an optionally substituted alkyl group;
R₈ is H, COR₉, CO₂R₉, Si(R₁₀)₃ or an optionally substituted alkyl group; and
R₉ and R₁₀ are each independently an optionally substituted alkyl group;
or the E and Z isomers thereof.

It is understood that the invention encompasses the cis and trans isomers or the E and Z configurations at the C═N bond of the hydrazyl group of the compound of formula I.

Moreover, unless stated otherwise, each alkyl, aryl or heteroaryl group is contemplated as being optionally substituted. An optionally substituted moiety may be substituted with one or more substituents. The substituent groups, which are optionally present, may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, oxo, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl or lower alkoxy groups e.g. C₁-C₄ alkyl or C₁-C₄ alkoxy groups. Unless otherwise specified, typically, 0-4 substituents may be present. When any of the foregoing substituents represents or contains an alkyl substituent group, this may be linear or branched and may contain up to 12 carbon atoms, preferably up to 6 carbon atoms, more preferably up to 4 carbon atoms.

In another embodiment, the term “optionally substituted” means that the moiety is substituted with 0-4 substituents independently selected from halogen atoms (e.g., Cl, Fl, Br), C₁-C₄alkyl (e.g., methyl, ethyl), C₁-C₄alkoxy (e.g., methoxy, ethoxy), C₁-C₄haloalkyl (e.g., CF₃ or CHF₂), C₁-C₄haloalkoxy (e.g., CF₃O), amino, nitro, carboxyl, alkylamino, dialkylamino or combinations thereof.

As used in the specification and claims, the ring “NR₅R₆” denotes an optionally substituted 5-7 membered heterocyclic ring. In one embodiment, “NR₅R₆” is an optionally substituted ring of formula V:

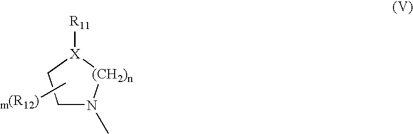

wherein m and n are each independently an integer of 1 to 3;
X is CH or N with the proviso that if X is N, n is 2 or 3; and
R₁₁ and each R₁₂ are independently selected from H, C₁-C₄alkyl, C₁-C₄ alkoxy, alkylamino, dialkylamino, COR₉, CO₂R₉, Si(R₁₀)₃ or an optionally substituted alkyl group, wherein R₉ and R₁₀ are as defined hereinabove.

As used herein, the term “alkyl” refers to a monovalent, straight- or branched-chain, saturated aliphatic hydrocarbon group having from 1 to 12 carbon atoms, preferably 1 to 6 carbon atoms, and more preferably 1 to 4 carbon atoms. Examples of alkyl moieties which are C₁-C₆alkyl groups include, but are not limited to, methyl (CH₃—); ethyl(CH₃CH₂—); propyl, e.g., n-propyl (CH₃CH₂CH₂—) and isopropyl ((CH₃)₂CH—); butyl, e.g., n-butyl (CH₃CH₂CH₂CH₂), tert-butyl ((CH₃)₃C—), isobutyl ((CH₃)₂CH₂CH₂—), and sec-butyl ((CH₃)(CH₃CH₂)CH—); pentyl, e.g., n-pentyl (CH₃CH₂CH₂CH₂CH₂—) and neopentyl ((CH₃)₃CCH₂—); and hexyl groups, e.g., n-hexyl (CH₃CH₂CH₂CH₂CH₂CH₂—), or the like. Specifically included within the definition of “alkyl” are those alkyl groups that are optionally substituted. Preferred alkyl substitutions include, but are not limited to, cyano, hydroxyl, a heterocyclyl, (e.g., NR₅R₆), halogen, phenyl, carbamoyl, oxo, alkoxy or aryloxy (e.g., benzyloxy or phenyloxy).

The term “alkoxy” as used herein, refers to the group alkyl-O— where the alkyl group is as defined herein. Specifically included within the definition of alkoxy are those alkoxy groups that are optionally substituted. Preferred alkoxy substitutions include, but are not limited to, halogen, amino, alkylamino, dialkylamino, phenyl, carbamoyl, oxo, or aryloxy (e.g., benzyloxy or phenyloxy), preferably dialkylamino.

“Amino” refers to the group —NH₂.

“Cyano” refers to the group —CN.

As used herein, the term “haloalkyl” designates a CₙH₂ₙ₊₁ group, e.g. having from 1 to 12 carbon atoms, preferably from 1 to 6 carbon atoms, more preferably from 1 to 4 carbon atoms, having from one to 2n+1 halogen atoms which may be the same or different. Examples of haloalkyl groups include CF₃, CH₂C₁, C₂H₃BrCl, C₃H₅F₂, or the like. A further example of a haloalkyl group is CHF₂.

The term “halogen”, or “halo”, as used herein, designates fluorine, chlorine, bromine, and iodine.

The term “aryl”, as used herein, refers to an aromatic carbocyclic moiety of up to 20 carbon atoms, e.g. from 6-20 carbon atoms, or from 6-14 carbon atoms, which may be a single ring (monocyclic) or multiple rings (bicyclic, up to three rings) fused together or linked covalently. Any suitable ring position of the aryl moiety may be covalently linked to the defined chemical structure. Examples of aryl moieties include, but are not limited to, phenyl, 1-naphthyl, 2-naphthyl, biphenyl, anthryl, phenanthryl, fluorenyl, indanyl, biphenylenyl, acenaphthenyl, acenaphthylenyl, and the like. In one embodiment, the aryl group is phenyl. In another embodiment, the aryl group is naphthyl. Preferred aryl substitutions include the following: halogen atoms (e.g., Cl, Fl, Br), C₁-C₄alkyl (e.g., methyl, ethyl), C₁-C₄alkoxy (e.g., methoxy, ethoxy), C₁-C₄haloalkyl (e.g., CF₃ or CHF₂), C₁-C₄haloalkoxy (e.g., CF₃O), amino, nitro, carboxyl, alkylamino, dialkylamino or combinations thereof.

The term “heteroaryl” as used herein designates an aromatic heterocyclic ring system, e.g. having from 5-20 ring members, or from 5-14 ring members, which may be a single ring (monocyclic) or multiple rings (bicyclic, up to three rings) fused together or linked covalently, provided that at least one of the rings is heteroaromatic. Preferably, heteroaryl is a 5- to 6-membered ring. The rings may contain from one to four hetero atoms selected from nitrogen, oxygen, or sulfur, wherein the nitrogen or sulfur atom(s) are optionally oxidized, or the nitrogen atom(s) are optionally quarternized. Any suitable ring position of the heteroaryl moiety may be covalently linked to the defined chemical structure. Examples of heteroaryl moieties include, but are not limited to, furan, thiophene, pyrrole, pyrazole, imidazole, oxazole, isoxazole, thiazole, isothiazole, oxadiazole, triazole, pyridine, pyrimidine, pyrazine, pyridazine, benzimidazole, benzoxazole, benzisoxazole, benzothiazole, benzofuran, benzothiophene, thianthrene, dibenzofuran, dibenzothiophene, indole, indazole, quinoline, isoquinoline, quinazoline, quinoxaline, purine, or the like. Preferred heteroaryl substitutions include the following: halogen atoms (e.g., Cl, Fl, Br), C₁-C₄alkyl (e.g., methyl, ethyl), C₁-C₄alkoxy (e.g., methoxy, ethoxy), C₁-C₄haloalkyl (e.g., CF₃ or CHF₂), C₁-C₄haloalkoxy (e.g., CF₃O), amino, nitro, carboxyl, alkylamino, dialkylamino or combinations thereof.

The term “cycloalkyl”, as used herein, refers to a monocyclic, bicyclic, tricyclic, fused, bridged, or spiro monovalent saturated hydrocarbon moiety of 3-14 carbon atoms. Cycloalkyl groups may be saturated or partially saturated. In one embodiment, “cycloalkyl” refers to cyclic alkyl groups of from 3 to 10 carbon atoms having single or multiple cyclic rings including fused, bridged, and spiro ring systems. The term “cycloalkyl” includes bicyclic alkyl groups, and bridged cycloalkyl groups which contain at least one carbon-carbon bond between two non-adjacent carbon atoms of the cycloalkyl ring. Examples of cycloalkyl moieties include, but are not limited to cyclopropyl, cyclobutyl, cyclopentyl, cyclohexyl, cycloheptyl, cyclooctyl, norbornyl, adamantyl, spiro[4.5]decanyl, or the like.

The term “heterocyclyl”, and the like as used herein, designates a 3 to 14 membered monovalent mono-, bi-, or tricyclic fused, bridged, or spiro ring system containing 1, 2 or 3 heteroatoms, which may be the same or different, selected from N, O or S and optionally containing one double bond. Exemplary of the heterocyclyl ring systems included in the term as designated herein are the following rings wherein X₁ is NR, O or S and R is H or an optional substituent as defined hereinabove.

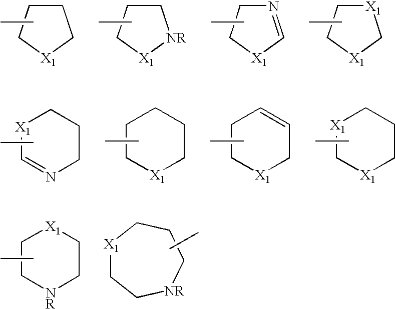

In one embodiment, the heterocyclyl ring is a 5- to 7-membered ring optionally containing an additional heteroatom according to formula V defined hereinabove. Preferred 5- to 7-membered ring substitutions include the following: C₁-C₄alkyl, C₁-C₄ alkoxy, alkylamino, dialkylamino, COR₉, CO₂R₉, Si(R₁₀)₃ or an optionally substituted alkyl group, wherein R₉ and R₁₀ are as defined hereinabove.

“Hydroxy” or “hydroxyl” refers to the group —OH.

“Nitro” refers to the group —NO₂.

“Deprotecting agent” as used herein refers to an agent capable of removing a protecting group from a nitrogen atom, and preferably includes acids, such as hydrochloric acid, hydrobromic acid, sulfuric acid, nitric acid, or bases, such as sodium hydroxide or potassium hydroxide.

“A copper containing catalyst” as used herein is meant to include any catalyst that contains copper in its (0), (I), and/or (II) oxidation state. Non-limiting examples include copper halide catalysts, such as Cu(I) catalysts, such as CuCl, CuBr and CuI.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

Unless otherwise indicated, the invention is not intended to include embodiments where an optional substituent is further substituted.

Similarly, it is understood that the above definitions are not intended to include impermissible substitution patterns (e.g., methyl substituted with 5 fluoro groups). Such impermissible substitution patterns are well known to the skilled artisan.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of another example, the term “5-7 membered ring” is specifically intended to individually disclose a ring having 5, 6, 7, 5-7, and 5-6 ring atoms.

Preferred compounds of formula I are those compounds wherein R₂ is an optionally substituted phenyl or optionally substituted naphthyl group. More preferred are those compounds of formula I where R₂ is an optionally substituted phenyl group. Another group of preferred compounds is those compounds of formula I wherein R₃ is H, NR₅R₆, or OR₇. In other preferred compounds R₄ is H. Particularly preferred are those compounds in which R₃ is H, NR₅R₆, or OR₇ and R₄ is H.

More preferred compounds of formula I are those compounds wherein R₂ is an optionally substituted phenyl or optionally substituted naphthyl group; R₃ is H, NR₅R₆, or OR₇; and R₄ is H. Another group of more preferred compounds are those compounds of formula I wherein R₃ is H, hydroxy, methoxy or an optionally substituted ring of formula Va:

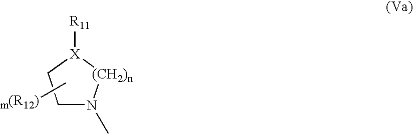

wherein m is 1 and n is 2;

### X is N; and

R₁₁ and each R₁₂ are independently selected from H, C₁-C₄alkyl, C₁-C₄ alkoxy, alkylamino, dialkylamino, COR₉, CO₂R₉, Si(R₁₀)₃ or an optionally substituted alkyl group, wherein R₉ and R₁₀ are as defined hereinabove.

Among the preferred compounds of the invention are:
(E)-N′-(2-chlorobenzylidene)phenylsulfonohydrazide;
(Z)-N′-(2-chlorobenzylidene)phenylsulfonohydrazide;
(E)-N′-(2-chlorobenzylidene)naphth-1-ylsulfonohydrazide;
(Z)-N′-(2-chlorobenzylidene)naphth-1-ylsulfonohydrazide;
(E)-N′-(2-chlorobenzylidene)naphth-2-ylsulfonohydrazide;
(Z)-N′-(2-chlorobenzylidene)naphth-2-ylsulfonohydrazide;
(E)-N′-(2-chloro-5-methoxybenzylidene)phenylsulfonohydrazide;
(Z)-N′-(2-chloro-5-methoxybenzylidene)phenylsulfonohydrazide;
(E)-N′-[2-chloro-6-(piperazin-1-yl)benzylidene]phenylsulfonohydrazide;
(Z)-N′-[2-chloro-6-(piperazin-1-yl)benzylidene]phenylsulfonohydrazide;
(E)-N′-[2-chloro-6-(piperazin-1-yl)benzylidene]naphth-2-ylsulfonohydrazide;
(Z)-N′-[2-chloro-6-(piperazin-1-yl)benzylidene]naphth-2-ylsulfonohydrazide;
(E)-N′-[2-chloro-6-(piperazin-1-yl)benzylidene]naphth-1-ylsulfonohydrazide;
(Z)-N′-[2-chloro-6-(piperazin-1-yl)benzylidene]naphth-1-ylsulfonohydrazide;
t-butyl 4-(3-chloro-2-{(Z)-[(phenylsulfonyl)hydrazono]methyl}-phenyl)piperazine-1-carboxylate; and
t-butyl 4-(3-chloro-2-{(E)-[(phenylsulfonyl)hydrazono]methyl}-phenyl)piperazine-1-carboxylate.

Advantageously, the present invention provides an efficient and effective process for the preparation of a compound of formula I which comprises reacting a 2-halobenzaldehyde of formula II with a sulfonylhydrazide of formula III at a temperature of about 35° C. to 120° C. optionally in the presence of a solvent. The process is shown in reaction scheme 1.

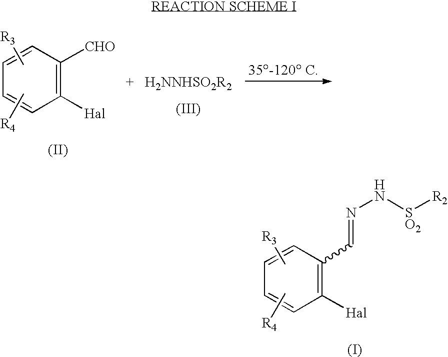

Solvents suitable for use in the process of the invention include 1,2-dichloro-ethane, acetonitrile, dioxane, isopropyl acetate, toluene, C₁-C₄alkanols, water, or the like, or a mixture thereof, preferably methanol or toluene.

In actual practice, one equivalent of a 2-halobenzaldehyde of formula II is admixed with at least one equivalent of a sulfonylhydrazide of formula III, optionally in the presence of a solvent such as methanol or toluene, to form a reaction mixture; the mixture is heated at about 35° to 120° C. until the reaction is complete; the reaction mixture is cooled and the reaction product is isolated by filtration or by removing the solvent under vacuum. The product may be the trans (E) form or the cis (Z) form or a mixture thereof. If so desired, the trans (E) form of the formula I product may be converted to the cis (Z) form by further heating at temperatures above the activation energy for isomerization.

Advantageously, the formula I compounds of the invention may be used in the manufacture of a 1-sulfonylindazole 5-HT6 ligand. Accordingly, the present invention also provides a process for the manufacture of a compound of formula IV

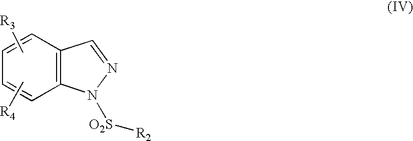

wherein
R₂ is an aryl or heteroaryl group each group optionally substituted;
R₃ and R₄ are each independently H, NR₅R₆, OR₇, or an optionally substituted alkyl group;
R₅ and R₆ are each independently H or an optionally substituted alkyl group or R₅ and R₆ may be taken together with the atom to which they are attached to form an optionally substituted 5- to 7-membered ring optionally containing an additional heteroatom selected from NR₈, O or S;
R₇ is H or an optionally substituted alkyl group;
R₈ is COR₉, CO₂R₉, Si(R₁₀)₃ or an optionally substituted alkyl group; and
R₉ and R₁₀ are each independently an optionally substituted alkyl group;
   which process comprises reacting a compound of formula I

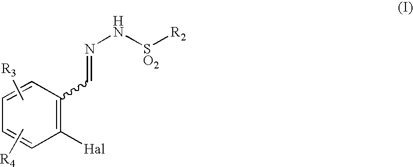

wherein R₂, R₃ and R₄ are as described hereinabove for formula IV and Hal is Cl, Br or I with a base in the presence of a copper containing catalyst in the presence of a solvent to form a compound of formula IV. The process is shown in reaction scheme II.

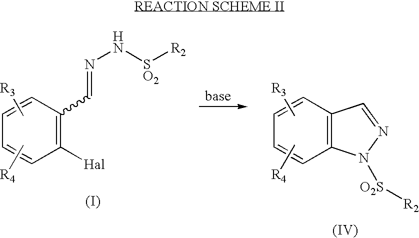

Bases suitable for use in the process of the invention include alkali metal carbonates such as K₂CO₃, Na₂CO₃, or the like; alkali metal bicarbonates such as KHCO₃, NaHCO₃, or the like; or any base known to be suitable for use in conventional synthetic procedures, preferably an alkali metal carbonate, more preferably K₂CO₃.

Solvents suitable for use in the inventive process include ethers such as tetrahydrofuran; amides such as dimethyl formamide; esters such as ethyl acetate; aromatic hydrocarbons such as toluene; aprotic solvents such as acetonitrile; or the like; preferably tetrahydrofuran or toluene.

In one embodiment of the invention, compounds of formula IV may be prepared by reacting compound of formula II

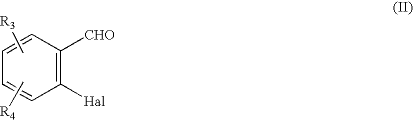

wherein Hal represents Cl, Br or I and R₃ and R₄ are as described hereinabove for formula IV with a sulfonylhydrazide compound of formula III

H₂NNHSO₂R₂ (III)

wherein R₂ is as described hereinabove for formula IV at a temperature of about 35° C. to 120° C. optionally in the presence of a first solvent to give a compound of formula I

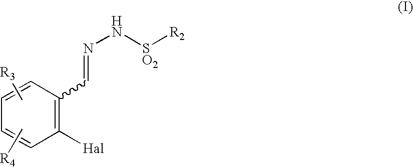

wherein Hal represents Cl, Br or I and R₂, R₃ and R₄ are as described hereinabove for formula IV; and reacting said formula I compound with a base in the presence of a copper containing catalyst in the presence of a second solvent to form a compound of formula IV.

The process is shown in reaction scheme III.

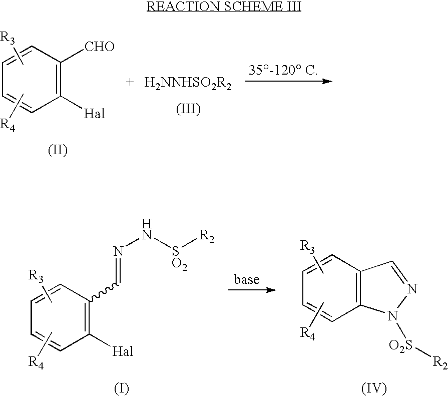

Solvents suitable for use as the first and second solvents include ethers such as tetrahydrofuran; amides such as dimethyl formamide; esters such as ethyl acetate; aromatic hydrocarbons such as toluene; aprotic solvents such as acetonitrile; water; or the like; or a mixture thereof, preferably toluene or a mixture of toluene and water.

Among the 1-arylsulfonylindazole compounds of formula IV which may be prepared by the process of the invention are those formula IV compounds wherein R₃ and R₄ are each independently H, NR₅R₆, OR₇, or an optionally substituted alkyl group. Another group of arylsulfonylindazole compounds of formula IV which may be prepared by the process of the invention is those formula IV compounds wherein R₃ is an optionally substituted piperazine ring. A further group of arylsulfonylindazole compounds of formula IV which may be prepared by the process of the invention is those formula IV compounds wherein R₂ is an optionally substituted phenyl or optionally substituted naphthyl group.

The invention further provides a process for the manufacture of a compound of formula IVa or a pharmaceutically acceptable salt thereof

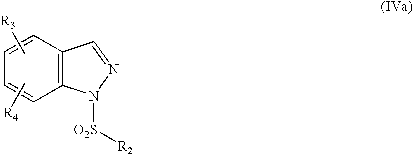

wherein
R₂ is an aryl or heteroaryl group each group optionally substituted;
R₃ and R₄ are each independently NR₅R₆ provided that at least one of R₃ and R₄ is NR₅R₆;
R₅ and R₆ are taken together with the atom to which they are attached to form an optionally substituted 5- to 7-membered ring optionally containing NR₈; and
R₈ is H;

which process comprises contacting a compound of formula IVb

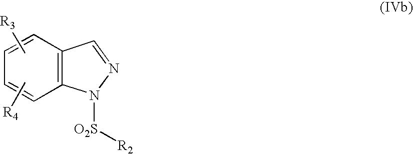

wherein
R₂ is an aryl or heteroaryl group each group optionally substituted;
R₃ and R₄ are each independently NR₅R₆ provided that at least one of R₃ and R₄ is NR₅R₆;
R₅ and R₆ are taken together with the atom to which they are attached to form an optionally substituted 5- to 7-membered ring optionally containing NR₈;
R₈ is COR₉, CO₂R₉, or Si(R₁₀)₃; and
R₉ and R₁₀ are each independently an optionally substituted alkyl group, with a deprotecting agent, thereby removing R₈ of formula IVb to form the compound of formula IVa; and optionally reacting with an acid to form the pharmaceutically acceptable salt of the compound of formula IVa.

In order to facilitate a further understanding of the invention, the following examples are presented primarily for the purpose of illustrating more specific details thereof. The invention is not to be limited thereby except as defined in the claims.

Unless otherwise noted, all parts are parts by weight. The terms THF and EtOAc designate tetrahydrofuran and ethyl acetate, respectively. The term “Boc” represents t-butoxycarbonyl. The terms HPLC and HNMR designate high performance liquid chromatography and proton nuclear magnetic resonance, respectively.

### EXAMPLES

### Example 1

### Preparation t-Butyl 4-(3-chloro-2-formylphenyl)piperazine-1-carboxylate

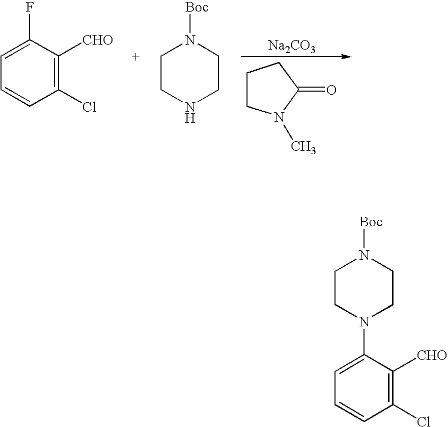

A mixture of 1-methyl-2-pyrrolidinone, 2-chloro-6-fluorobenzaldehyde (0.100 kg, 0.63 mol) and sodium carbonate (0.125 kg, 1.01 mol) was heated at 110° C., treated dropwise, over a 20 min period, with a solution of N-Boc-piperazine (0.123 kg, 0.662 mol) in 1-methyl-2-pyrrolidinone. The reaction mixture was stirred for 4.5 h at 110° C., cooled to 70-80° C., treated dropwise, over a 40 min. period, with water while maintaining the temperature at 70-80° C., cooled to 45-50° C. and filtered. The filtercake was washed with water and dried under nitrogen to give the title compound as a yellow solid, 181 g (88.3% yield), 94% purity by HPLC, mp 100-103° C.

### Example 2

### Preparation t-Butyl 4-(3-chloro-2-formylphenyl)piperazine-1-carboxylate

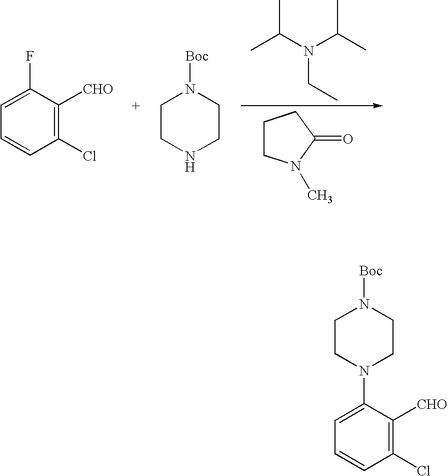

A mixture of 1-methyl-2-pyrrolidinone, 2-chloro-6-fluorobenzaldehyde, 95% (40 g, 0.25 mol), N,N-diisopropylethylamine (48.9 g, 1.5 eq), N-Boc-piperazine (49.3 g, 0.27 mol) was heated at 90°-100° C. for 11 h, cooled to 0-10° C., treated dropwise, over a 20 min. period, with water:acetone (2:1) mixture, stirred for 15 min at 0-10° C. and filtered. The wet filtercake was washed with water and dried under nitrogen to give the title compound as a yellow solid, 78.5 g (96.0% yield), 91% purity by HPLC, mp 100-103° C.

### Example 3

### Preparation of t-Butyl 4-(3-Chloro-2-{(E)-[(phenylsulfonyl)hydrazono]-methyl}phenyl)piperazine-1-carboxylate

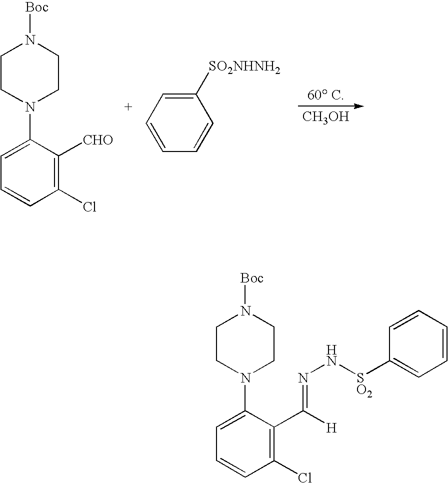

A mixture of benzenesulfonyl hydrazide (6.3 g, 0.04 mol), and t-butyl 4-(3-chloro-2-formylphenyl)piperazine-1-carboxylate (11.3 g, 0.03 mol) in methanol was heated at 60° C. for 30 min, treated dropwise, over a 20 min. period, with water while maintaining the temperature between 50-60° C., stirred for 1 h at 0°-10° C. and filtered. The wet filtercake was washed with heptane and dried under nitrogen to give the title compound as an off-white solid, 16.6 g, 80% purity by HPLC, mp 162-165° C. The solid was dissolved in a 1:2 mixture of heptane:isopropanol, heated at 75° C. for 5 min, cooled to room temperature. stirred for 1 h and filtered. The filtercake was air-dried to give the title compound as white solid, 9.8 g (63.5% yield), 99% purity by HPLC, mp 168-171° C., identified by HNMR and mass spectral analyses.

### Example 4

### Preparation of t-Butyl 4-(3-Chloro-2-{(Z)-[(phenylsulfonyl)hydrazono]methyl}-phenyl)piperazine-1-carboxylate

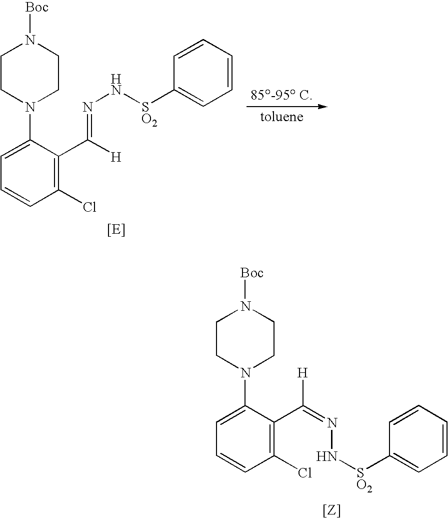

A solution of t-butyl 4-(3-chloro-2-{(E)-[(phenylsulfonyl)hydrazono]methyl}phenyl)piperazine-1-carboxylate (100 g, 0.209 mol) in toluene was stirred at 85-95° C. for 2 h and concentrated in vacuo at 30-80° C. to give a red oil. The oil was dissolved in isopropanol, heated at 50-60° C., treated dropwise, over a 15 min. period, with heptane, cooled to 0°-10° C. for 30 min, stirred for another 30 min. and filtered. The wet cake was washed with isopropanol:heptane (2:1) and dried under vacuum at 40-50° C. to give the title compound as an off-white solid, 54.3 g (54% yield), 98.4% purity by HPLC, mp 139-141° C., identified by HNMR and mass spectral analyses.

### Example 5

### Preparation of t-Butyl 4-[1-(Phenylsulfonyl)-1H-indazol-4-yl]piperazine-1-carboxylate

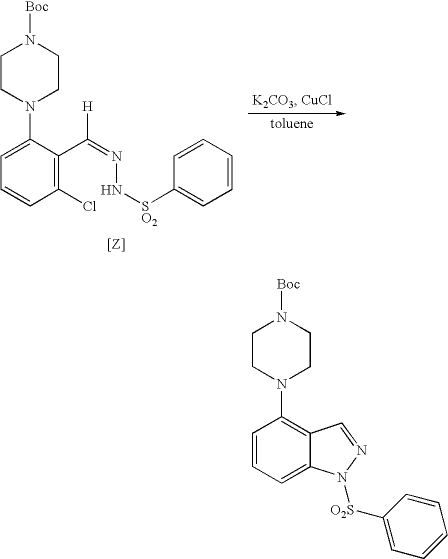

A mixture of t-butyl 4-(3-chloro-2-{(Z)-[(phenylsulfonyl)hydrazono]methyl}phenyl)piperazine-1-carboxylate (25 g, 0.052 mol), copper (I) chloride (1.3 g, 0.013 mol) and powder potassium carbonate, anhydrous, (18 g, 0.13 mol) in toluene was heated at 85°-90° C. for 6 h, cooled to 10°-25° C., washed with NH₄OH and water, concentrated under reduced pressure at 45°-55° C. to a volume of 45-60 mL, heated to 60°-65° C., treated dropwise, over a 15 min. period, with heptane, cooled to 0°-10° C., stirred for 2 h and filtered. The wet filtercake was washed with heptane and dried under vacuum at 40°-50° C. to give the title compound as a white to off-white solid, 16.1 g (85.5% yield), 98.6% purity by HPLC, mp 130° C.

### Example 6

### Preparation of t-Butyl 4-[1-(Phenylsulfonyl)-1H-indazol-4-yl]piperazin-1-carboxylate

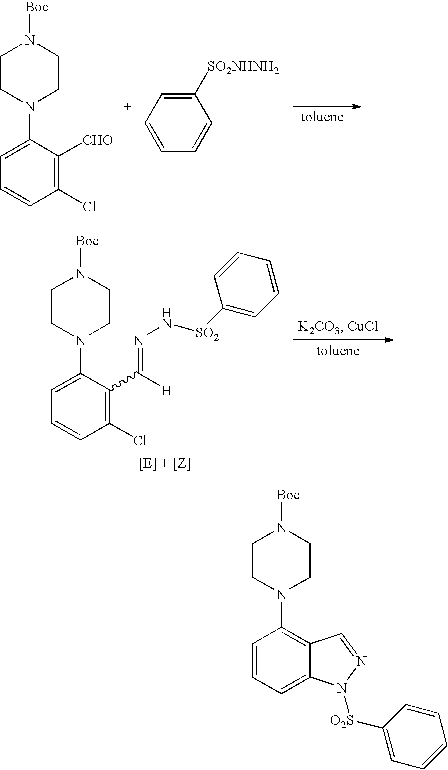

A mixture of benzenesulfonyl hydrazide (0.058 kg, 0.314 mol) in toluene at 35° to 45° C. was treated over a 1 h period with a solution of t-butyl 4-(3-chloro-2-formylphenyl)piperazine-1-carboxylate (0.100 kg, 0.308 mol) in toluene, heated at 85° to 95° C. for 3.5 h to give about a 3:1 cis:trans isomer ratio of t-butyl 4-(3-chloro-2-phenylsulfonyl)hydrazono]methyl}phenyl)piperazine-1-carboxylate. The reaction mixture was cooled to 50-60° C., treated sequentially with copper (I) chloride (0.0015 kg, 0.0154 mol) as slurry in water and an aqueous solution of K₂CO₃ (0.053 kg, 0.38 mol) over a 30 min. period, stirred for 1 h at 50°-60° C., heated at 75°-85° C. for 2 h, cooled to 10°-25° C., and washed with NH₄OH and water and concentrated under vacuum at 45°-55° C. to a volume of 0.3 L, heated to 60°-70° C., treated with heptane, cooled to 0°-10° C., stirred for 2 h and filtered. The wet filtercake was washed with heptane and dried under vacuum at 40°-50° C. to give the title compound as white to off-white solid, 77.7 g (57% yield), 81% purity by HPLC, mp 130° C.

### Example 7

### Preparation of 1-(Phenylsulfonyl)-4-(1′-piperazinyl)-1H-indazole Hydrochloride

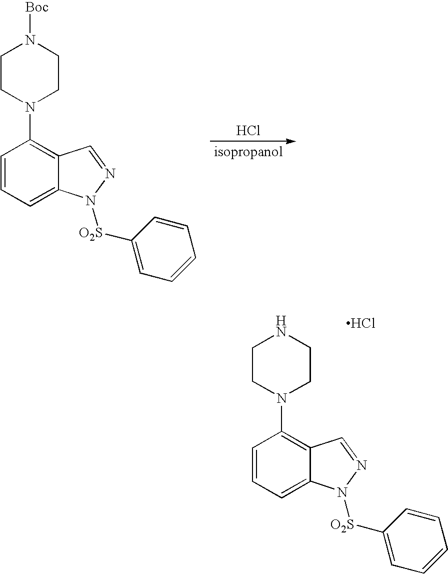

A solution of t-butyl 4-[1-(phenylsulfonyl)-1H-indazol-4-yl]piperazine-1-carboxylate (0.25 kg, 0.57 mol) in isopropanol was treated dropwise, over a 20 min. period, with 4N isopropanolic HCl (1.7 mol HCl) at room temperature. The reaction mixture was heated at 50°-55° C. for 5.5 h, cooled to 0°-10° C. and filtered. The wet filtercake was washed with isopropanol and dried under vacuum at 40°-50° C. to give the title compound as an off-white solid, 217 g (86.2% yield), 99.5% purity by HPLC, mp 217° C.

What is claimed is:

1. A compound of formula I 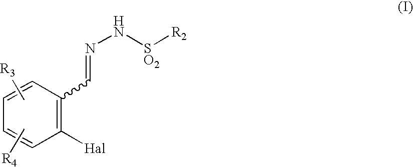
wherein
Hal is Cl, Br or I;
R₂ is an optionally substituted aryl or optionally substituted heteroaryl group;
R₃ and R₄ are each independently H, NR₅R₆, OR₇, or an optionally substituted alkyl group;
R₅ and R₆ are each independently H or an optionally substituted alkyl group or R₅ and R₆ may be taken together with the atom to which they are attached to form an optionally substituted 5- to 7-membered ring optionally containing an additional heteroatom selected from NR₈, O or S;
R₇ is H or an optionally substituted alkyl group;
R₈ is H, COR₉, CO₂R₉, Si(R₁₀)₃ or an optionally substituted alkyl group; and
R₉ and R₁₀ are each independently an optionally substituted alkyl group; or
the E and Z isomers thereof.

2. The compound according to claim 1 wherein R₂ is an optionally substituted phenyl or optionally substituted naphthyl group

3. The compound according to claim 1 wherein R₃ is H, NR₅R₆, or OR₇ and R₄ is H.

4. The compound according to claim 3 wherein R₂ is an optionally substituted phenyl or optionally substituted naphthyl group.

5. The compound according to claim 4 wherein R₃ is H, hydroxy, methoxy, or an optionally substituted ring of formula Va: 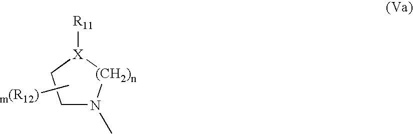
wherein m is 1 and n is 2;
X is N; and
R₁₁ and each R₁₂ are independently selected from H, C₁-C₄alkyl, C₁-C₄ alkoxy, alkylamino, dialkylamino, COR₉, CO₂R₉, Si(R₁₀)₃ or an optionally substituted alkyl group, wherein R₉ and R₁₀ are as defined hereinabove.

6. The compound according to claim 1 selected from the group consisting of:
(E)-N′-(2-chlorobenzylidene)phenylsulfonohydrazide;
(E)-N′-(2-chlorobenzylidene)naphth-1-ylsulfonohydrazide;
(E)-N′-(2-chlorobenzylidene)naphth-2-ylsulfonohydrazide;
(E)-N′-(2-chloro-5-methoxybenzylidene)phenylsulfonohydrazide;
(E)-N′-[2-chloro-6-(piperazin-1-yl)benzylidene]phenylsulfonohydrazide;
(E)-N′-[2-chloro-6-(piperazin-1-yl)benzylidene]naphth-2-ylsulfonohydrazide;
(E)-N′-[2-chloro-6-(piperazin-1-yl)benzylidene]naphth-1-ylsulfonohydrazide; and
t-Butyl 4-(3-Chloro-2-{(E)-[(phenylsulfonyl)hydrazono]methyl}-phenyl)piperazine-1-carboxylate.

7. The compound according to claim 1 selected from the group consisting of:
(Z)-N′-(2-chlorobenzylidene)phenylsulfonohydrazide;
(Z)-N′-(2-chlorobenzylidene)naphth-1-ylsulfonohydrazide;
(Z)-N′-(2-chlorobenzylidene)naphth-2-ylsulfonohydrazide;
(Z)-N′-(2-chloro-5-methoxybenzylidene)phenylsulfonohydrazide;
(Z)-N′-[2-chloro-6-(piperazin-1-yl)benzylidene]phenylsulfonohydrazide;
(Z)-N′-[2-chloro-6-(piperazin-1-yl)benzylidene]naphth-2-ylsulfonohydrazide;
(Z)-N′-[2-chloro-6-(piperazin-1-yl)benzylidene]naphth-1-ylsulfonohydrazide; and
t-Butyl 4-(3-Chloro-2-{(Z)-[(phenylsulfonyl)hydrazono]methyl}-phenyl)piperazine-1-carboxylate.

8. A process for the manufacture of a compound of formula I 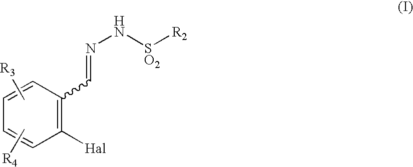
wherein
Hal is Cl, Br or I;
R₂ is an optionally substituted aryl or optionally substituted heteroaryl group;
R₃ and R₄ are each independently H, NR₅R₆, OR₇, or an optionally substituted alkyl group;
R₅ and R₆ are each independently H or an optionally substituted alkyl group or R₅ and R₆ may be taken together with the atom to which they are attached to form an optionally substituted 5- to 7-membered ring optionally containing an additional heteroatom selected from NR₈, O or S;
R₇ is H or an optionally substituted alkyl group;
R₈ is H, COR₉, CO₂R₉, Si(R₁₀)₃ or an optionally substituted alkyl group; and
R₉ and R₁₀ are each independently an optionally substituted alkyl group;
which process comprises reacting a compound of formula II 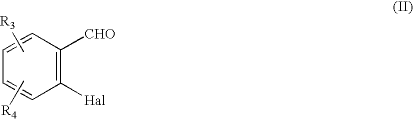
wherein Hal, R₃ and R₄ are as described hereinabove; with a sulfonylhydrazide compound of formula III
H₂NNHSO₂R₂ (III)
wherein R₂ is as described hereinabove; at a temperature of about 35° C. to 120° C., optionally in the presence of a solvent to form a compound of formula I.

9. The process according to claim 8 wherein the solvent is 1,2-dichloro-ethane, acetonitrile, dioxane, isopropyl acetate, toluene or a C₁-C₄alkanol.

10. The process according to claim 9 wherein the solvent is toluene.

11. A process for the manufacture of a compound of formula IV 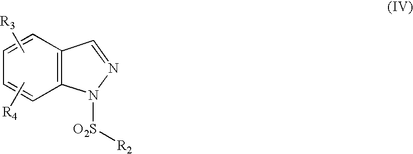
wherein
R₂ is an aryl or heteroaryl group each group optionally substituted;
R₃ and R₄ are each independently H, NR₅R₆, OR₇, or an optionally substituted alkyl group;
R₅ and R₆ are each independently H or an optionally substituted alkyl group or R₅ and R₆ may be taken together with the atom to which they are attached to form an optionally substituted 5- to 7-membered ring optionally containing an additional heteroatom selected from NR₈, O or S;
R₇ is H or an optionally substituted alkyl group;
R₈ is COR₉, CO₂R₉, Si(R₁₀)₃ or an optionally substituted alkyl group; and
R₉ and R₁₀ are each independently an optionally substituted alkyl group;
which process comprises reacting a compound of formula I 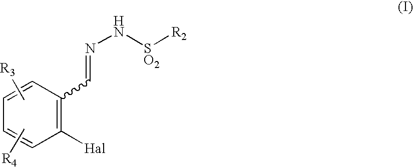
wherein R₂, R₃ and R₄ are as described hereinabove; and Hal is Cl, Br or I with a base in the presence of a copper containing catalyst in the presence of a solvent to form a compound of formula IV.

12. The process according to claim 11 wherein the base is an alkali metal carbonate or an alkali metal bicarbonate.

13. The process according to claim 12 wherein the base is an alkali metal carbonate.

14. The process according to claim 12 wherein the solvent is tetrahydrofuran, dimethyl formamide, ethyl acetate, toluene, or acetonitrile, or a mixture thereof.

15. The process according to claim 14 wherein the solvent is tetrahydrofuran and the base is K₂CO₃.

16. A process for the manufacture of a compound of formula IV 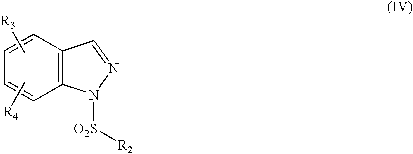
wherein
R₂ is an aryl or heteroaryl group each group optionally substituted;
R₃ and R₄ are each independently H, NR₅R₆, OR₇, or an optionally substituted alkyl group;
R₅ and R₆ are each independently H or an optionally substituted alkyl group or R₅ and R₆ may be taken together with the atom to which they are attached to form an optionally substituted 5- to 7-membered ring optionally containing an additional heteroatom selected from NR₈, O or S;
R₇ is H or an optionally substituted alkyl group;
R₈ is COR₉, CO₂R₉, Si(R₁₀)₃ or an optionally substituted alkyl group; and
R₉ and R₁₀ are each independently an optionally substituted alkyl group;
which process comprises reacting compound of formula II 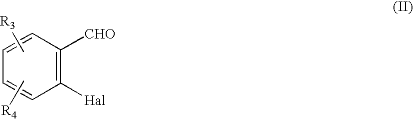
wherein Hal represents Cl, Br or I and R₃ and R₄ are as described hereinabove; with a sulfonylhydrazide compound of formula III
H₂NNHSO₂R₂ (III)
wherein R₂ is as described hereinabove; at a temperature of about 35° C. to 120° C., optionally in the presence of a first solvent to give a compound of formula I 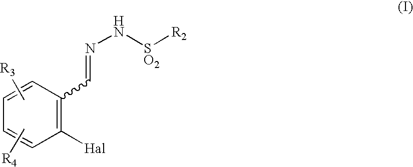
wherein Hal represents Cl, Br or I and R₂, R₃ and R₄ are as described hereinabove; and reacting said formula I compound with a base in the presence of a copper containing catalyst in the presence of a second solvent.

17. The process according to claim 16 wherein the first solvent is 1,2-dichloro-ethane, acetonitrile, dioxane, isopropyl acetate, toluene or a C₁-C₄alkanol.

18. The process according to claim 17 wherein the second solvent is tetrahydrofuran, dimethyl formamide, ethyl acetate, toluene, or acetonitrile, or a mixture thereof

19. The process according to claim 18 wherein the base is an alkali metal carbonate or an alkali metal bicarbonate.

20. The process according to claim 16 for the manufacture of a formula IV compound wherein R₂ is an optionally substituted phenyl or optionally substituted naphthyl group.

21. The process according to claim 20 wherein R₄ is H; and R₃ is an optionally substituted ring of formula Va: 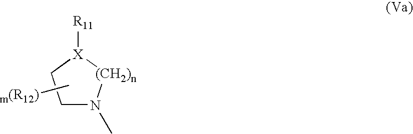
wherein m is 1 and n is 2;
X is N; and
R₁₁ and each R₁₂ are independently selected from H, C₁-C₄alkyl, C₁-C₄ alkoxy, alkylamino, dialkylamino, COR₉, CO₂R₉, Si(R₁₀)₃ or an optionally substituted alkyl group, wherein R₉ and R₁₀ are as defined hereinabove.

22. A process for the manufacture of a compound of formula IVa or a pharmaceutically acceptable salt thereof 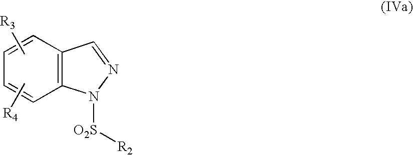
wherein
R₂ is an aryl or heteroaryl group each group optionally substituted;
R₃ and R₄ are each independently NR₅R₆ provided that at least one of R₃ and R₄ is NR₅R₆;
R₅ and R₆ are taken together with the atom to which they are attached to form an optionally substituted 5- to 7-membered ring optionally containing NR₈; and
R₈ is H;
which process comprises contacting a compound of formula IVb 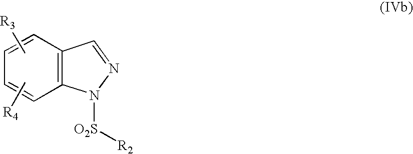
wherein
R₂ is an aryl or heteroaryl group each group optionally substituted;
R₃ and R₄ are each independently NR₅R₆ provided that at least one of R₃ and R₄ is NR₅R₆;
R₅ and R₆ are taken together with the atom to which they are attached to form an optionally substituted 5- to 7-membered ring optionally containing NR₈;
R₈ is COR₉, CO₂R₉, or Si(R₁₀)₃; and
R₉ and R₁₀ are each independently an optionally substituted alkyl group,
with a deprotecting agent, thereby removing R₈ of formula IVb to form the compound of formula IVa; and optionally reacting with an acid to form the pharmaceutically acceptable salt of the compound of formula IVa.
